(19) United States
(12) Patent Application Publication
(10) Pub. No.: US 2016/0060187 A1
(43) Pub. Date: Mar. 3, 2016

(54) Treatment of Off-Gas in the Production of Para-Xylene by the Methylation of Toluene and/or Benzene

(71) Applicant: ExxonMobil Chemical Patents Inc., Baytown, TX (US)

(72) Inventors: James L. Kendall, Humble, TX (US); Gregory E. Yeo, Houston, TX (US); Robert D. Strack, Houston, TX (US)

(21) Appl. No.: 14/801,144

(22) Filed: Jul. 16, 2015

Related U.S. Application Data
(60) Provisional application No. 62/041,717, filed on Aug. 26, 2014.

(51) Int. Cl. C07C 2/86; B01J 19/24

(52) U.S. Cl. CPC C07C 2/864; B01J 19/24; B01J 2219/24; C07C 2529/06

(57) ABSTRACT

A process for removing contaminants from an off-gas stream generated by the methylation of toluene and/or benzene by methanol to produce para-xylene. The treated off-gas stream, which contains C₄₋ hydrocarbons, may be further processed in an olefins plant/process to yield valuable light alkanes and olefins.

### PRIORITY CLAIM

This application claims priority to and the benefit of U.S. Provisional Application No. 62/041,717, filed Aug. 26, 2014, the disclosure of which is incorporated herein by reference in its entirety.

### FIELD OF THE INVENTION

This invention relates to a process for treating off-gas produced in the production of para-xylene by the alkylation of benzene and/or toluene with methanol.

### BACKGROUND OF THE INVENTION

Of the xylene isomers, para-xylene is of particular value since it is useful in the manufacture of terephthalic acid, which is an intermediate in the manufacture of synthetic fibers and resins. Today, para-xylene is commercially produced by hydrotreating of naphtha (catalytic reforming), steam cracking of naphtha or gas oil, and toluene disproportionation.

One problem with most existing processes for producing xylenes is that they produce a thermodynamic equilibrium mixture of ortho (o)-, meta (m)- and para (p)-xylenes, in which the para-xylene concentration is typically only about 24 wt %. Thus, separation of para-xylene from such mixtures tends to require superfractionation and multistage refrigeration steps. Such processes involve high operating and capital costs and result in only limited yields. There is therefore a continuing need to provide processes which are highly selective for the production of para-xylene.

It is well-known to manufacture xylenes by the alkylation of toluene and/or benzene with methanol, and, in particular, to selectively make para-xylene (PX) product using zeolite catalyst. See, for instance, U.S. Pat. Nos. 4,002,698; 4,356,338; 4,423,266; 5,675,047; 5,804,690; 5,939,597; 6,028,238; 6,046,372; 6,048,816; 6,156,949; 6,423,879; 6,504,072; 6,506,954; 6,538,167; and 6,642,426. The terms “para-xylene selectivity”, “para-selective”, and the like, means that para-xylene is produced in amounts greater than is present in a mixture of xylene isomers at thermodynamic equilibrium, which at ordinary processing temperatures is about 24 mol %. Para-xylene selectivity is highly sought after because of the economic importance of para-xylene relative to meta- and ortho-xylene. Although each of the xylene isomers have important and well-known end uses, para-xylene is currently the most economically valuable.

In the process, typically toluene and/or benzene are alkylated with methanol, in the presence of a suitable catalyst, to form xylenes in a reactor in a system illustrated schematically in FIG. 1, wherein a feed comprising reactants enter fluid bed reactor 11 via conduit 1 and effluent comprising product exits through conduit 5, and the catalyst circulates between fluid bed reactor 11, apparatus 12, which strips fluid from the catalyst, and catalyst regenerator 13, via conduits 2, 3, and 4, respectively. Water is typically co-fed with toluene and methanol to minimize toluene coking in the feed lines and methanol self-decomposition. Other side reactions include the formation of light olefins, light paraffins, as reactions that convert para-xylenes to other xylene isomers or heavier aromatics.

Although toluene methylation, and particularly the para-selective toluene methylation process of U.S. Pat. No. 6,504,072, provides an attractive route to para-xylene, the process inevitably produces significant quantities of light (C₄−) gas. These gaseous by-products include olefins, particularly ethylene, propylene and butylenes; alkanes, such as methane, ethane, propane and butanes, which may be recovered and purified to increase their value above fuel value. However, also present in the off-gas stream are contaminants, such as nitrogen (N₂), carbon monoxide (CO), and nitrogen oxides (NOx).

U.S. Patent Publication No. 2014/0100402 discloses a process for the recovery of olefins from the methylation of toluene effluent. The disclosed process utilizes a cryogenic separation unit to separate ethylene and propylene from the off-gas stream. Cryogenic processing can cause nitrogen oxides in the gas to form nitrogen oxide salts and gums in the presence of ammonia and/or reactive hydrocarbons, which are unstable at elevated temperatures. Without proper mitigation facilities, the nitrogen oxide salts and gums in a cryogenic unit can explode as the temperature of equipment increases above its normal operating condition. Thus, the nitrogen oxides and other contaminants in the off-gas stream must be removed prior to the recovery of olefins.

### SUMMARY OF THE INVENTION

The present invention provides a process for removing contaminants from an off-gas stream generated by the methylation of toluene and/or benzene by methanol to produce para-xylene. The treated off-gas stream, which contains C₄₋ hydrocarbons, may be further processed in an olefins plant/process to yield valuable light alkanes and olefins.

The invention resides in a process for producing para-xylene, the process comprising (a) contacting benzene and/or toluene with methanol in the presence of an alkylation catalyst under conditions effective to produce an alkylation effluent comprising xylenes and a by-product mixture comprising water, dimethyl ether, gaseous C₄− hydrocarbons, and contaminants; (b) separating the alkylation effluent into a first fraction containing xylenes and a second fraction containing the by-product mixture; (c) recovering para-xylene from the first fraction; and (d) treating the second fraction to remove the contaminants In a preferred embodiment, the second fraction is treated by an absorber demethanizer, which counter-currently contacts the second fraction with a C₂-C₆ hydrocarbon absorbent, preferably a C₃-C₄ hydrocarbon. Ethylene and propylene may be recovered from the treated second fraction.

The invention also provides an apparatus for the production of para-xylene comprising an alkylation reactor for the methylation of toluene and/or benzene with methanol in the presence of an alkylation catalyst to produce an alkylation effluent comprising xylenes and a by-product mixture comprising water, dimethyl ether, C₄− hydrocarbons, and contaminants; a separation system for the separation of the alkylation effluent into a first fraction containing xylenes and a second fraction containing the by-product mixture; an absorber demethanizer for removing contaminants from the second fraction in which an absorbent counter-currently contacts the second fraction; and a second separation system for the recovery of the absorbent.

### BRIEF DESCRIPTION OF THE DRAWINGS

FIG. 1 is a schematic of a reactor system including reactor and regenerator and some associated auxiliary devices and transfer piping per se known in the art.

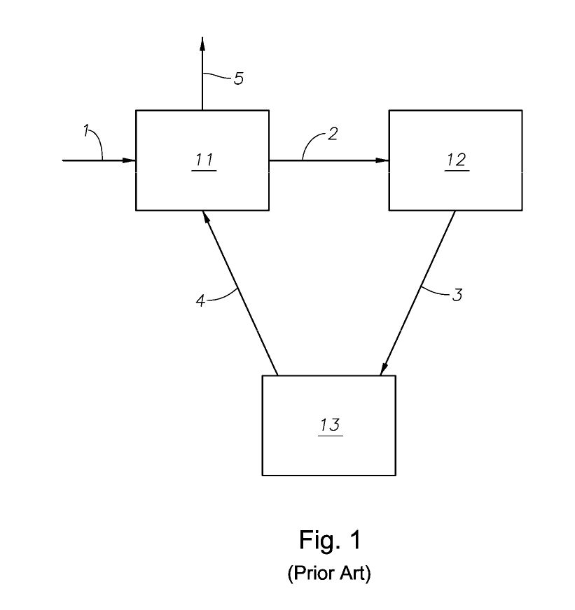

FIG. 2 is a flow diagram of a process for treating the off-gas stream from a methanol/toluene alkylation process, according to one example of the present application.

### DETAILED DESCRIPTION OF THE EMBODIMENTS

Described herein is a process for producing para-xylene by the catalytic alkylation of benzene and/or toluene with methanol. The alkylation process produces a para-rich mixture of xylene isomers, together with water and some light organic by-products, particularly dimethyl ether and C₄− olefinic hydrocarbons. The off-gas stream, containing the C₄₋ hydrocarbons, also contains contaminants such as nitrogen, carbon monoxide, and nitrogen oxides. The present process provides a process of removing the contaminants from the off-gas stream so that at least the olefins from these light by-products may be recovered for uses other than as fuel.

### Alkylation Process

The alkylation process employed herein can employ any aromatic feedstock comprising benzene and/or toluene, although in general it is preferred that the aromatic feed contains at least 90 wt %, especially at least 99 wt %, of toluene. Similarly, although the composition of the methanol-containing feed is not critical, it is generally desirable to employ feeds containing at least 90 wt %, especially at least 99 wt %, of methanol.

The catalyst employed in the alkylation process is generally a porous crystalline material and, in one preferred embodiment, is a porous crystalline material having a Diffusion Parameter for 2,2 dimethylbutane of about 0.1-15 sec⁻¹ when measured at a temperature of 120° C. and a 2,2 dimethylbutane pressure of 60 torr (8 kPa).

As used herein, the Diffusion Parameter of a particular porous crystalline material is defined as D/r²×10⁶, wherein D is the diffusion coefficient (cm²/sec) and r is the crystal radius (cm). The diffusion parameter can be derived from sorption measurements provided the assumption is made that the plane sheet model describes the diffusion process.

Thus, for a given sorbate loading Q, the value Q/Q_{eq}, where Q_{eq} is the equilibrium sorbate loading, is mathematically related to (Dt/r²)^{1/2} where t is the time (sec) required to reach the sorbate loading Q. Graphical solutions for the plane sheet model are given by J. Crank in “The Mathematics of Diffusion”, Oxford University Press, Ely House, London, 1967.

The porous crystalline material is preferably a medium-pore size aluminosilicate zeolite. Medium pore zeolites are generally defined as those having a pore size of about 5 to about 7 Angstroms, such that the zeolite freely sorbs molecules such as n-hexane, 3-methylpentane, benzene, and para-xylene. Another common definition for medium pore zeolites involves the Constraint Index test which is described in U.S. Pat. No. 4,016,218, which is incorporated herein by reference. In this case, medium pore zeolites have a Constraint Index of about 1-12, as measured on the zeolite alone without the introduction of oxide modifiers and prior to any steaming to adjust the diffusivity of the catalyst. In addition to the medium-pore size aluminosilicate zeolites, other medium pore acidic metallosilicates, such as silicoaluminophosphates (SAPOs), can be used in the present process.

Particular examples of suitable medium pore zeolites include ZSM-5, ZSM-11, ZSM-12, ZSM-22, ZSM-23, ZSM-35, and ZSM-48, with ZSM-5 and ZSM-11 being particularly preferred. In one embodiment, the zeolite employed in the process of the invention is ZSM-5 having a silica to alumina molar ratio of at least 250, as measured prior to any treatment of the zeolite to adjust its diffusivity.

Zeolite ZSM-5 and the conventional preparation thereof are described in U.S. Pat. No. 3,702,886. Zeolite ZSM-11 and the conventional preparation thereof are described in U.S. Pat. No. 3,709,979. Zeolite ZSM-12 and the conventional preparation thereof are described in U.S. Pat. No. 3,832,449. Zeolite ZSM-23 and the conventional preparation thereof are described in U.S. Pat. No. 4,076,842. Zeolite ZSM-35 and the conventional preparation thereof are described in U.S. Pat. No. 4,016,245. ZSM-48 and the conventional preparation thereof are taught by U.S. Pat. No. 4,375,573. The entire disclosures of these U.S. patents are incorporated herein by reference.

The medium pore zeolites described above are preferred for the present process since the size and shape of their pores favor the production of para-xylene over the other xylene isomers. However, conventional forms of these zeolites have Diffusion Parameter values in excess of the 0.1-15 sec⁻¹ range desired for the present process. Nevertheless, the required diffusivity can be achieved by severely steaming the zeolite so as to effect a controlled reduction in the micropore volume of the catalyst to not less than 50%, and preferably 50-90%, of that of the unsteamed catalyst. Reduction in micropore volume is monitored by measuring the n-hexane adsorption capacity of the zeolite, before and after steaming, at 90° C. and 75 torr n-hexane pressure.

Steaming to achieve the desired reduction in the micropore volume of the porous crystalline material can be effected by heating the material in the presence of steam at a temperature of at least about 950° C., preferably about 950 to about 1075° C., and most preferably about 1000 to about 1050° C. for about 10 minutes to about 10 hours, preferably from 30 minutes to 5 hours.

To effect the desired controlled reduction in diffusivity and micropore volume, it may be desirable to combine the porous crystalline material, prior to steaming, with at least one oxide modifier, preferably selected from oxides of the elements of Groups IIA, IIIA, IIIB, IVA, VA, VB and VIA of the Periodic Table (IUPAC version). Conveniently, said at least one oxide modifier is selected from oxides of boron, magnesium, calcium, lanthanum and preferably phosphorus. In some cases, it may be desirable to combine the porous crystalline material with more than one oxide modifier, for example a combination of phosphorus with calcium and/or magnesium, since in this way it may be possible to reduce the steaming severity needed to achieve a target diffusivity value. The total amount of oxide modifier present in the catalyst, as measured on an elemental basis, may be between about 0.05 and about 20 wt %, such as between about 0.1 and about 10 wt %, based on the weight of the final catalyst.

Where the modifier includes phosphorus, incorporation of modifier in the alkylation catalyst is conveniently achieved by the methods described in U.S. Pat. Nos. 4,356,338; 5,110,776; 5,231,064 and 5,348,643, the entire disclosures of which are incorporated herein by reference. Treatment with phosphorus-containing compounds can readily be accomplished by contacting the porous crystalline material, either alone or in combination with a binder or matrix material, with a solution of an appropriate phosphorus compound, followed by drying and calcining to convert the phosphorus to its oxide form. Contact with the phosphorus-containing compound is generally conducted at a temperature of about 25° C. and about 125° C. for a time between about 15 minutes and about 20 hours. The concentration of the phosphorus in the contact mixture may be between about 0.01 and about 30 wt %.

Representative phosphorus-containing compounds which may be used to incorporate a phosphorus oxide modifier into the catalyst of the invention include derivatives of groups represented by PX₃, RPX₂, R₂PX, R₃P, X₃PO, (XO)₃PO, (XO)₃P, R₃P═O, R₃P═S, RPO₂, RPS₂, RP(O)(OX)₂, RP(S)(SX)₂, R₂P(O)OX, R₂P(S)SX, RP(OX)₂, RP(SX)₂, ROP(OX)₂, RSP(SX)₂, (RS)₂PSP(SR)₂, and (RO)₂POP(OR)₂, where R is an alkyl or aryl, such as phenyl radical, and X is hydrogen, R, or halide. These compounds include primary, RPH₂, secondary, R₂PH, and tertiary, R₃P, phosphines such as butyl phosphine, the tertiary phosphine oxides, R₃PO, such as tributyl phosphine oxide, the tertiary phosphine sulfides, R₃PS, the primary, RP(O)(OX)₂, and secondary, R₂P(O)OX, phosphonic acids such as benzene phosphonic acid, the corresponding sulfur derivatives such as RP(S)(SX)₂ and R₂P(S)SX, the esters of the phosphonic acids such as dialkyl phosphonate, (RO)₂P(O)H, dialkyl alkyl phosphonates, (RO)₂P(O)R, and alkyl dialkylphosphinates, (RO)P(O)R₂; phosphinous acids, R₂POX, such as diethylphosphinous acid, primary, (RO)P(OX)₂, secondary, (RO)₂POX, and tertiary, (RO)₃P, phosphites, and esters thereof such as the monopropyl ester, alkyl dialkylphosphinites, (RO)PR₂, and dialkyl alkyphosphinite, (RO)₂PR, esters. Corresponding sulfur derivatives may also be employed including (RS)₂P(S)H, (RS)₂P(S)R, (RS)P(S)R₂, R₂PSX, (RS)P(SX)₂, (RS)₂PSX, (RS)₃P, (RS)PR₂, and (RS)₂PR. Examples of phosphite esters include trimethylphosphite, triethylphosphite, diisopropylphosphite, butylphosphite, and pyrophosphites such as tetraethylpyrophosphite. The alkyl groups in the mentioned compounds preferably contain one to four carbon atoms.

Other suitable phosphorus-containing compounds include ammonium hydrogen phosphate, the phosphorus halides such as phosphorus trichloride, bromide, and iodide, alkyl phosphorodichloridites, (RO)PCl₂, dialkylphosphoro-chloridites, (RO)₂PCl, dialkylphosphinochloroidites, R₂PCl, alkyl alkylphosphonochloridates, (RO)(R)P(O)Cl, dialkyl phosphinochloridates, R₂P(O)Cl, and RP(O)Cl₂. Applicable corresponding sulfur derivatives include (RS)PCl₂, (RS)₂PCl, (RS)(R)P(S)Cl, and R₂P(S)Cl.

Particular phosphorus-containing compounds include ammonium phosphate, ammonium dihydrogen phosphate, diammonium hydrogen phosphate, diphenyl phosphine chloride, trimethylphosphite, phosphorus trichloride, phosphoric acid, phenyl phosphine oxychloride, trimethylphosphate, diphenyl phosphinous acid, diphenyl phosphinic acid, diethylchlorothiophosphate, methyl acid phosphate, and other alcohol-P₂O₅ reaction products.

Representative boron-containing compounds, which may be used to incorporate a boron oxide modifier into the catalyst of the invention, include boric acid, trimethylborate, boron oxide, boron sulfide, boron hydride, butylboron dimethoxide, butylboric acid, dimethylboric anhydride, hexamethylborazine, phenyl boric acid, triethylborane, diborane, and triphenyl boron.

Representative magnesium-containing compounds include magnesium acetate, magnesium nitrate, magnesium benzoate, magnesium propionate, magnesium 2-ethylhexoate, magnesium carbonate, magnesium formate, magnesium oxylate, magnesium bromide, magnesium hydride, magnesium lactate, magnesium laurate, magnesium oleate, magnesium palmitate, magnesium salicylate, magnesium stearate, and magnesium sulfide.

Representative calcium-containing compounds include calcium acetate, calcium acetylacetonate, calcium carbonate, calcium chloride, calcium methoxide, calcium naphthenate, calcium nitrate, calcium phosphate, calcium stearate, and calcium sulfate.

Representative lanthanum-containing compounds include lanthanum acetate, lanthanum acetylacetonate, lanthanum carbonate, lanthanum chloride, lanthanum hydroxide, lanthanum nitrate, lanthanum phosphate, and lanthanum sulfate.

The porous crystalline material employed in the process of the invention may be combined with a variety of binder or matrix materials resistant to the temperatures and other conditions employed in the process. Such materials include active and inactive materials such as clays, silica and/or metal oxides such as alumina The latter may be either naturally occurring or in the form of gelatinous precipitates or gels including mixtures of silica and metal oxides. Use of a material which is active, tends to change the conversion and/or selectivity of the catalyst and hence is generally not preferred. Inactive materials suitably serve as diluents to control the amount of conversion in a given process so that products can be obtained economically and orderly without employing other means for controlling the rate of reaction. These materials may be incorporated into naturally occurring clays, e.g., bentonite and kaolin, to improve the crush strength of the catalyst under commercial operating conditions. Said materials, i.e., clays, oxides, etc., function as binders for the catalyst. It is desirable to provide a catalyst having good crush strength because in commercial use it is desirable to prevent the catalyst from breaking down into powder-like materials. These clay and/or oxide binders have been employed normally only for the purpose of improving the crush strength of the catalyst.

Naturally occurring clays which can be composited with the porous crystalline material include the montmorillonite and kaolin family, which families include the subbentonites, and the kaolins commonly known as Dixie, McNamee, Georgia, and Florida clays or others in which the main mineral constituent is halloysite, kaolinite, dickite, nacrite, or anauxite. Such clays can be used in the raw state as originally mined or initially subjected to calcination, acid treatment, or chemical modification.

In addition to the foregoing materials, the porous crystalline material can be composited with a porous matrix material such as silica-alumina, silica-magnesia, silica-zirconia, silica-thoria, silica-beryllia, silica-titania, as well as ternary compositions such as silica-alumina-thoria, silica-alumina-zirconia, silica-alumina-magnesia, and silica-magnesia-zirconia.

The relative proportions of porous crystalline material and inorganic oxide matrix vary widely, with the content of the former ranging from about 1 to about 90% by weight and more usually, particularly when the composite is prepared in the form of beads, in the range of about 2 to about 80 wt % of the composite.

The alkylation process can be conducted in any known reaction vessel but generally the methanol and aromatic feeds are contacted with the catalyst described above with the catalyst particles being disposed in one or more fluidized beds. Each of the methanol and aromatic feeds can be injected into the fluidized catalyst in a single stage. However, in one embodiment, the methanol feed is injected in stages into the fluidized catalyst at one or more locations downstream from the location of the injection of the aromatic reactant into the fluidized catalyst. For example, the aromatic feed can be injected into a lower portion of a single vertical fluidized bed of catalyst, with the methanol being injected into the bed at a plurality of vertically spaced intermediate portions of the bed and the product being removed from the top of the bed. Alternatively, the catalyst can be disposed in a plurality of vertically spaced catalyst beds, with the aromatic feed being injected into a lower portion of the first fluidized bed and part of the methanol being injected into an intermediate portion of the first bed and part of the methanol being injected into or between adjacent downstream catalyst beds.

The conditions employed in the alkylation stage of the present process are not narrowly constrained but, in the case of the methylation of toluene, generally include the following ranges: (a) temperature between about 500 and about 700° C., such as between about 500 and about 600° C.; (b) pressure of between about 1 atmosphere and about 1000 psig (between about 100 and about 7000 kPa), such as between about 10 psig and about 200 psig (between about 170 and about 1480 kPa); (c) moles toluene/moles methanol (in the reactor charge) of at least about 0.2, such as from about 0.2 to about 20; and (d) a weight hourly space velocity (“WHSV”) for total hydrocarbon feed to the reactor(s) of about 0.2 to about 1000, such as about 0.5 to about 500 for the aromatic reactant, and about 0.01 to about 100 for the combined methanol reagent stage flows, based on total catalyst in the reactor(s).

### Product Treatment and Recovery

The product of the reaction between the methanol and toluene and/or benzene is an alkylation effluent comprising para-xylene and other xylene isomers, water vapor, unreacted toluene and/or benzene, unreacted methanol, phenolic impurities, and a variety of light gas by-products, such as C₄− hydrocarbons, including light olefins and contaminants such as nitrogen, nitrogen oxides, carbon monoxide, carbon dioxide, and oxygenates such as ethanal and dimethyl ether. The alkylation effluent will also generally contain some C₉+ aromatic by-products. In addition, where the process is conducted in a fluidized catalyst bed, the alkylation effluent will contain some entrained solid catalyst and catalyst fines. Thus the effluent, which is generally in the vapor phase, leaving the (final) fluidized bed reactor is generally passed through an integral cyclone separator to remove some of the entrained catalyst solids and return them to the alkylation reactor.

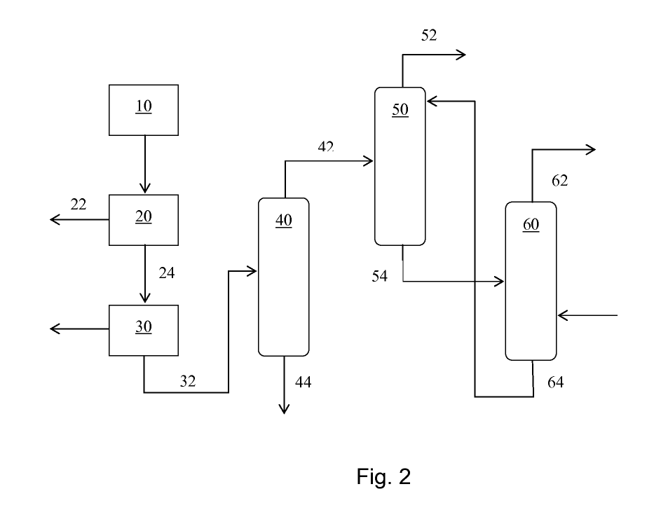

With reference to FIG. 2, the alkylation effluent leaves the alkylation reactor system 10 at a high temperature, typically between about 500 and about 600° C. and initially may be passed through a heat exchanger so that the waste heat in the effluent stream may be recovered and used to heat other process stream(s). It is, however, preferred that any initial cooling of the product stream is limited so as to keep the effluent vapors well above the dew point, typically about 240° F. (116° C.).

Following further cooling, the effluent vapor stream is fed to a separation system 20, which may comprise one or more fractionation columns, where the alkylation effluent is separated into a first fraction 22 containing xylenes and a second fraction 24 containing the by-product mixture. Further separations to recover the unreacted methanol, unreacted benzene and/or toluene, heavy (C₉+) by-products and other by-products are possible and within the skill of one in the art. Para-xylene is recovered from the first fraction 22, typically by fractional crystallization or selective adsorption (not shown).

In the present process, the second fraction 24, containing the light (C₄−) hydrocarbons, is treated by a treatment system 30 to recover at least the valuable olefinic component of the stream. In embodiments, the second fraction 24 is subjected to compression in the treatment system 30. The compressed stream then goes through a series of wash steps, such as a methanol wash to remove oxygenates, a water wash to remove methanol, and a caustic wash to remove carbon dioxide. The stream may then be dried to remove water, such as with a molecular sieve drier or by washing with methanol, which itself has preferably been dried to remove water, such as with a molecular sieve drier.

In embodiments, the dried by-product mixture 32 is then sent to a fractionation tower 40 primarily to remove dimethyl ether from the light olefins, so as to minimize the impact of dimethyl ether on olefins recovery equipment. Dimethyl ether can also be deleterious to a later-recovered propylene product by negatively impacting propylene in downstream processes such as polymerization. The fractionation tower acts to fractionate the dried by-product mixture into an overhead stream 42, containing at least some, and preferably most, of the C₃− hydrocarbons, and almost all of the dimethyl ether and C₄+ hydrocarbons as a liquid bottoms stream 44. For example, ethylene and at least about 80 wt %, preferably at least about 90 wt %, of the propylene, and about 67 wt % of the propane from the fractionation column are recovered in the overhead stream, while nearly 100 wt % of the dimethyl ether and nearly 100 wt % of C₄+ hydrocarbons are removed in the liquid bottoms stream. The overhead vapor stream 42 from the fractionation tower 40, which generally comprises less than about 100 ppm dimethyl ether, preferably 20 ppm or less by weight, more preferably 1 ppm or less by weight is sent to a contaminant removal system.

In embodiments, the second fraction is treated to remove the contaminants from the off-gas stream. In preferred embodiments, the overhead vapor stream 42, containing hydrogen, methane, ethane, ethylene, propane, propylene, nitrogen, carbon monoxide, and nitrogen oxides, is fed to an absorber demethanizer 50. An absorber demethanizer is effective at removing the nitrogen, carbon monoxide, and nitrogen oxide contaminants from the off-gas stream without the risks associated with a cryogenic system. The absorber demethanizer operates by contacting the off-gas stream counter-currently with a hydrocarbon absorbent. As the absorbent travels down the column and interacts with the off-gas stream traveling up the column, at least some, and preferably most, of the C₂₊ hydrocarbons in the off-gas stream are absorbed by the absorbent and exit the absorber demethanizer in a bottoms stream 54. The hydrogen, methane, nitrogen, carbon monoxide, and nitrogen oxides, along with a small percentage of the C₂ and C₃ hydrocarbons, exit the absorber demethanizer as an overhead stream 52, which may be used as fuel. The absorber demethanizer may be equipped with a reboiler to minimize the amount of methane and contaminants in the bottoms stream 54.

The hydrocarbon absorbent may be selected from a C₂-C₆ hydrocarbon, preferably a C₃-C₅ hydrocarbon, and more preferably a C₃-C₄ hydrocarbon or mixture thereof. Examples of suitable hydrocarbon absorbents are ethane, propane, propylene, n-butane, isobutane, n-butylene, isobutylene, 1-butene, cis-butene, trans-butene, butadiene, and pentane. The absorbent must be free from contaminants that would affect the downstream operations and free from water that could cause hydrate formation in the absorber demethanizer. In a preferred embodiment, the absorbent used is a C₃ or C₄ hydrocarbon or a mixture thereof For example, the absorbent may be propylene, a C₄ hydrocarbon, or a mixture of propylene and C₄ hydrocarbons, such as propylene and butenes. In a more preferred embodiment, the absorbent used is a C₄ hydrocarbon or a mixture of C₄ hydrocarbons, as less C₄ hydrocarbons are lost to fuel in the absorber demethanizer relative to C₃ hydrocarbon absorbents. For example, the absorbent may be n-butane, isobutane, isobutylene, 1-butene, cis-butene, trans-butene butadiene, or a mixture thereof The absorbent used may also contain an amount of other components that do not materially affect the characteristics of the absorbent. Thus, a C₃ hydrocarbon absorbent may also contain an amount of C₂ hydrocarbons, but the amount of C₂ hydrocarbons does not affect the characteristics of the C₃ hydrocarbon absorbent. Likewise, a C₄ hydrocarbon absorbent may also contain an amount of C₃ hydrocarbons, but the amount of C₃ hydrocarbons does not affect the characteristics of the C₄ hydrocarbon absorbent.

A person skilled in the art can determine the optimum operating temperature and pressure for the absorber demethanizer based on the hydrocarbon absorbent used, available refrigerant and desired economic recovery. As a general rule of safety, the higher the operating temperature, the lesser the risk of formation of nitrogen oxide salts and gums.

The bottoms stream 54 containing the absorbent, C₂ and C₃ hydrocarbons are sent to a separation system 60, typically a fractionation column or columns, where the absorbent is separated for recirculation through the absorber demethanizer. The separation system 60 may be selected by one skilled in the art based upon the absorbent used. Preferably, the separation system 60 is a single fractionation column to concentrate the hydrocarbon absorbent for reuse. In an embodiment in which a C₄ hydrocarbon or a mixture of C₄ hydrocarbons is used as the absorbent, the separation system 60 is a debutanizer. The bottoms stream 54 from the absorber demethanizer enters the separation system 60, which separates the C₂ and C₃ hydrocarbons from the C₄ hydrocarbon absorbent. The C₂ and C₃ hydrocarbons exit the separation system 60 as an overhead stream 62 and are sent to further processing in an olefins plant/process to yield valuable light alkanes and olefins. The absorbent leaves the separation system 60 as a bottoms stream 64 for recirculation through the absorber demethanizer 50. Additional absorbent, to offset the losses to fuel, may be introduced into the separation system 60.

While the present invention has been described and illustrated by reference to particular embodiments, those of ordinary skill in the art will appreciate that the invention lends itself to variations and modifications not necessarily illustrated herein without departing from the spirit and scope of the invention. Accordingly, it is not intended that the scope of the claims appended hereto be limited to the examples and descriptions set forth herein but rather that the claims be construed as encompassing all the features of patentable novelty which reside herein, including all features which would be treated as equivalents thereof by those skilled in the art to which this disclosure pertains.

Trade names used herein are indicated by a ™ symbol or ® symbol, indicating that the names may be protected by certain trademark rights, e.g., they may be registered trademarks in various jurisdictions. All patents and patent applications, test procedures (such as ASTM methods, UL methods, and the like), and other documents cited herein are fully incorporated by reference to the extent such disclosure is not inconsistent with this invention and for all jurisdictions in which such incorporation is permitted. When numerical lower limits and numerical upper limits are listed herein, ranges from any lower limit to any upper limit are contemplated. The term “comprising” is synonymous with the term “including”. Likewise whenever a composition, a element or a group of components is preceded with the transitional phrase “comprising,” it is understood that we also contemplate the same composition or group of components with transitional phrases “consisting essentially of,” “consisting of,” “selected from the group of consisting of,” or “is” preceding the recitation of the composition, component, or components, and vice versa.

What we claim:

1. A process for producing para-xylene, the process comprising:
(a) contacting toluene and/or benzene with methanol in the presence of an alkylation catalyst under conditions effective to produce an alkylation effluent comprising xylenes and a by-product mixture comprising water, dimethyl ether, C₄− hydrocarbons, and contaminants;
(b) separating the alkylation effluent into a first fraction containing xylenes and a second fraction containing the by-product mixture;
(c) recovering para-xylene from the first fraction; and
(d) treating the second fraction to remove the contaminants.

2. The process of claim 1, wherein the para-xylene is recovered by fractional crystallization or selective adsorption.

3. The process of claim 1, wherein the treating (d) is accomplished by counter-currently contacting the second fraction with a C₂-C₆ hydrocarbon absorbent in an absorber demethanizer.

4. The process of claim 3, wherein the absorbent consists essentially of a C₃ or C₄ hydrocarbon or a mixture thereof.

5. The process of claim 3, wherein the absorbent consists essentially of a C₄ hydrocarbon or a mixture thereof.

6. The process of claim 3, wherein the treating (d) produces an overhead stream comprising hydrogen, methane and contaminants and a bottoms stream comprising C₂₊ hydrocarbons and absorbent, and further comprising:
(e) separating the absorbent from the differing C₂₊ hydrocarbons.

7. The process of claim 6, wherein the absorbent recovered in the separating (e) is recirculated to the treating (d).

8. The process of claim 6, wherein the separating (e) is accomplished by a single fractionation column.

9. The process of claim 1, further comprising:
(f) recovering ethylene and propylene from the treated second fraction.

10. The process of claim 1, wherein the toluene is provided in a feedstream containing at least about 90 wt % toluene.

11. The process of claim 1, wherein the alkylation catalyst is a porous crystalline material having a Diffusion Parameter for 2,2 dimethylbutane of about 0.1-15 sec⁻¹ when measured at a temperature of 120° C. and a 2,2 dimethylbutane pressure of 60 torr (8 kPa).

12. The process of claim 11, wherein the alkylation catalyst is a medium-pore size aluminosilicate zeolite selected from the group consisting of ZSM-5, ZSM-11, ZSM-12, is ZSM-22, ZSM-23, ZSM-35, and ZSM-48, optionally composited with an inorganic oxide matrix.

13. The process of claim 1, wherein a methanol feed is injected in stages into the alkylation catalyst at one or more locations downstream from the location of injection of the toluene.

14. A process for producing para-xylene by the methylation of toluene and/or benzene with methanol in the presence of an alkylation catalyst under conditions effective to produce an alkylation effluent comprising xylenes and an off-gas stream containing contaminants, said process comprising the treatment of the off-gas stream with an absorber demethanizer to remove the contaminants.

15. The process of claim 14, wherein the treatment is accomplished by counter-currently contacting the second fraction with a C₂-C₆ hydrocarbon absorbent.

16. The process of claim 15, wherein the absorbent consists essentially of a C₃ or C₄ hydrocarbon or a mixture thereof.

17. The process of claim 15, wherein the absorbent consists essentially of a C₄ hydrocarbon or a mixture thereof.

18. An apparatus for the production of para-xylene comprising:
(a) an alkylation reactor for the methylation of toluene and/or benzene with methanol in the presence of an alkylation catalyst to produce an alkylation effluent comprising xylenes and a by-product mixture comprising water, dimethyl ether, C₄− hydrocarbons, and contaminants;
(b) a separation system for the separation of the alkylation effluent into a first fraction containing xylenes and a second fraction containing the by-product mixture;
(c) an absorber demethanizer for removing contaminants from the second fraction in which a C₂-C₆ hydrocarbon absorbent counter-currently contacts the second fraction; and
(d) a second separation system for the recovery of the absorbent.

19. The apparatus of claim 18, further comprising a para-xylene recovery system.

20. The apparatus of claim 18, wherein the second separation system comprises a single fractionation column.

21. The apparatus of claim 18, wherein the absorbent consists essentially of a C₃ or C₄ hydrocarbon or a mixture thereof.
